(19) United States
(12) Patent Application Publication
(10) Pub. No.: US 2017/0197063 A1
(43) Pub. Date: Jul. 13, 2017

(54) Balloon Inside a Guide Catheter

(71) Applicant: Mudassar Ahmed, Minneapolis, MN (US)

(72) Inventor: Mudassar Ahmed, Minneapolis, MN (US)

(21) Appl. No.: 14/991,925

(22) Filed: Jan. 9, 2016

(51) Int. Cl. A61M 25/10; A61M 25/01

(52) U.S. Cl. CPC A61M 25/10; A61M 25/0172; A61M 25/104

(57) ABSTRACT

Techniques are described for a medical device that includes a securing balloon inside of a guide catheter. A catheter comprising a proximal portion, a distal portion, and an expanding member located at the distal portion, said distal portion having an inner wall and an outer wall, said expanding member attached to said distal portion at the inner wall.

### BACKGROUND

Vascular equipment for treating cardiovascular diseases comes mainly in two forms: “rapid exchange” or “over the wire.” In both cases, the physician may use a guiding device, such as a guide wire, to controllably reach the lesion or area to be treated. Once the guide wire is in position, the physician may need to pass one or more catheters, tubular devices, and/or medical devices along the guide wire to the treatment area. Physicians may use fluoroscopy techniques for visualizing the treatment area, as well as visualizing the location and movement of catheters, wires, and medical devices used during coronary procedures.

In the case of rapid exchange systems, the physician is able to insert or remove the catheter or tubular device along the guide wire for almost the entire length of the said catheter without having to fluoroscopically confirm absence of guide wire movement. This is because part of the guide wire is always exposed through the proximal end of a rapid exchange system and can be manually fixed to prevent movement.

One common application of rapid exchange catheters is during coronary angioplasty, which refers to the use of an inflatable balloon to increase the blood flow through a stenosis (i.e., partially blocked section of a blood vessel feeding the heart). Coronary angioplasty techniques may include a physician inserting a guiding catheter into a patient's blood vessel, for example the femoral artery. The guiding catheter is advanced toward the heart through the patient's arterial system and used to engage one of the coronary arteries. The physician may then insert a guide wire into the guiding catheter until the distal end of the guide wire exits the guiding catheter and enters the coronary artery, positioning the guide wire across the stenosis to be treated in the coronary artery. The physician must use caution not to inadvertently move the guide wire out of position. Finally, the physician advances a balloon catheter along the guide wire until the balloon exits the guiding catheter and is positioned across the stenosis. The physician then inflates the balloon to treat the stenosis, deflates the balloon, and removes the balloon catheter without disturbing the placement of either the guide wire of the guiding catheter.

Physicians frequently need to exchange balloon catheters during a single coronary angioplasty procedure. For example, if a stenosis blocks most of the blood flow through a vessel, the physician may first need to use a small balloon to increase the size of the opening through the stenosis, and then use a larger balloon to further increase the opening. Another example of a catheter exchange is when a physician first uses a balloon catheter to open a lumen and a second catheter to deploy a stent.

During catheter exchanges, it is important that the guide wire not move from its initial position. When using rapid exchange systems, this is done by the operator or assistant, manually securing the proximal exposed part of the guide wire during catheter exchanges. Despite this, there can occasionally be movement of the wire.

The problem of preventing wire movement becomes an issue when using over the wire systems rather than rapid exchange systems. Over the wire systems have a wire lumen that runs the entire length of the catheter, rather than a short segment as in rapid exchange systems. The use of an over the wire system offers several advantages over rapid exchange systems for example, if the physician wants to use a different guide wire during a case, the over the wire catheter can be taken down to the end of the wire, the wire removed and a new wire with perhaps different characteristics inserted without the wire having to recross diseased portions of the vessel. Another advantage of an over the wire system is the ability to inject contrast or medications through the distal tip directly into the target vasculature.

Despite these advantages, over the wire systems are not used as frequently as rapid exchange systems, because of the need for long (e.g., 300 cm) guide wires. Catheter exchanges over long wires are more difficult, need more fluoroscopy (increasing radiation exposure for the patient and staff), and make it more likely that the guide wire could lose position during the case, which increases case time, case complexity and potentially affects patient outcomes.

### SUMMARY

In one example, a medical device may comprise a catheter comprising a proximal portion, a distal portion, and an expanding member located at the distal portion, said distal portion having an inner wall and an outer wall, said expanding member attached to said distal portion at the inner wall. In another example, a method may comprise inserting a catheter in a vessel of a patient, wherein the catheter has an inner and outer wall inserting medical equipment in the catheter, and inflating a balloon attached to a first portion of the inner wall of the catheter. In other examples, a medical equipment securing system may comprise a catheter that has a proximal portion, a distal portion, and an expandable balloon located at the distal portion, said balloon being arranged for expanding from a contracted state to an expanded state, said balloon being adapted for expansion from a distal end thereof, the balloon having an inflation port at a distal portion thereof.

The details of one or more examples of the disclosure are set forth in the accompanying drawings and the description below. Other features, objects, and advantages will be apparent from the description and drawings, and from the claims.

### BRIEF DESCRIPTION OF DRAWINGS

FIG. 1A is a conceptual diagram illustrating an example of a catheter with a balloon attached to the inner wall of the catheter, in accordance with one or more techniques of the present disclosure.

FIG. 1B is a conceptual diagram illustrating another view of an example of a balloon attached to the catheter, in accordance with one or more techniques of the present disclosure.

FIG. 2A is a conceptual diagram illustrating an example of an inflated balloon that is attached to the inner wall of the catheter, in accordance with one or more techniques of the present disclosure.

FIG. 2B is a conceptual diagram illustrating an example of another view of the inflated balloon attached to the inner wall of the catheter and securing medical equipment against the inner wall, in accordance with one or more techniques of the present disclosure.

FIG. 3 is a flow diagram illustrating example steps of the method for using a catheter with a balloon attached to the inner wall of the catheter, in accordance with one or more techniques of the present disclosure.

### DETAILED DESCRIPTION

In general, this disclosure is directed to a catheter that secures a guide wire during vascular disease procedures. In some examples, the catheter is a guide catheter that has an inflatable balloon attached to the inner wall of the guide catheter. Once a physician or technician places the guide catheter, the physician may insert medical equipment, such as guide wires, microcatheters, and/or other medical devices necessary for a specific type of coronary or vascular procedure into the guide catheter inserted in the vascular system of the patient. During the procedure, the physician may need to remove or exchange the inserted microcatheters, guide wires, or medical devices, depending on the type of procedure and the challenges during the procedure. In some examples, the physician may use additional medical devices during the procedure. Improving a guide catheter by attaching a balloon to the catheter's interior wall, allows one to use or deliver any microcatheter, stent, balloon or other device with less cumbersome coronary wires, e.g. 190 cm (or “short”), and switch the coronary wire as needed (with the support of a microcatheter) without risk of losing wire position.

When the physician inflates the balloon attached to the inner wall of the guide catheter, the balloon expands to fill the hollow interior of the guide catheter. As the balloon inflates, the balloon contacts the guide wire positioned in the guide catheter pushing the guide wire outward from the center of the guide catheter and against the inner wall. The outward pressure of the inflated balloon limits that motion of the guide wire, securing it in place so a physician may exchange, remove, or add any medical devices or equipment needed during the procedure over the guide wire without the latter moving.

In addition to guide wires, such a balloon attached to the distal portion of the guiding catheter may also be used to prevent movement and secure the position of other equipment within the guide e.g. micro catheters or balloon catheters. Rather than have the physician exchange guide wires or lose position, such medical devices may reduce the time of the procedure as the physician does not have to replace wires nor does the guide wire move from the desired placement. Consequently, the shorter procedure time also reduces radiation exposure during fluoroscopic procedures when medical equipment is being delivered and positioned in the arterial system of the patient, particularly in the coronary arteries. Additionally, such medical devices may also improve procedure quality as the reduced motion of the guide wire/microcatheter reduces the risk of perforations or other complications. The advantages of just using 150-200 cm wires are that they are easier to handle by the physicians, technicians and other operators, less likely to be accidentally dislodged. The presence of a device that actively secures a guide wire within a guiding catheter may allow physicians to use over the wire equipment as easily as rapid exchange equipment, increasing therapeutic choices for physicians and patients, making the procedure safer by decreasing procedure time, decreasing radiation (as fluoroscopy may not be needed as much to confirm that the wire had not moved) and increasing procedural success rates and patient outcomes.

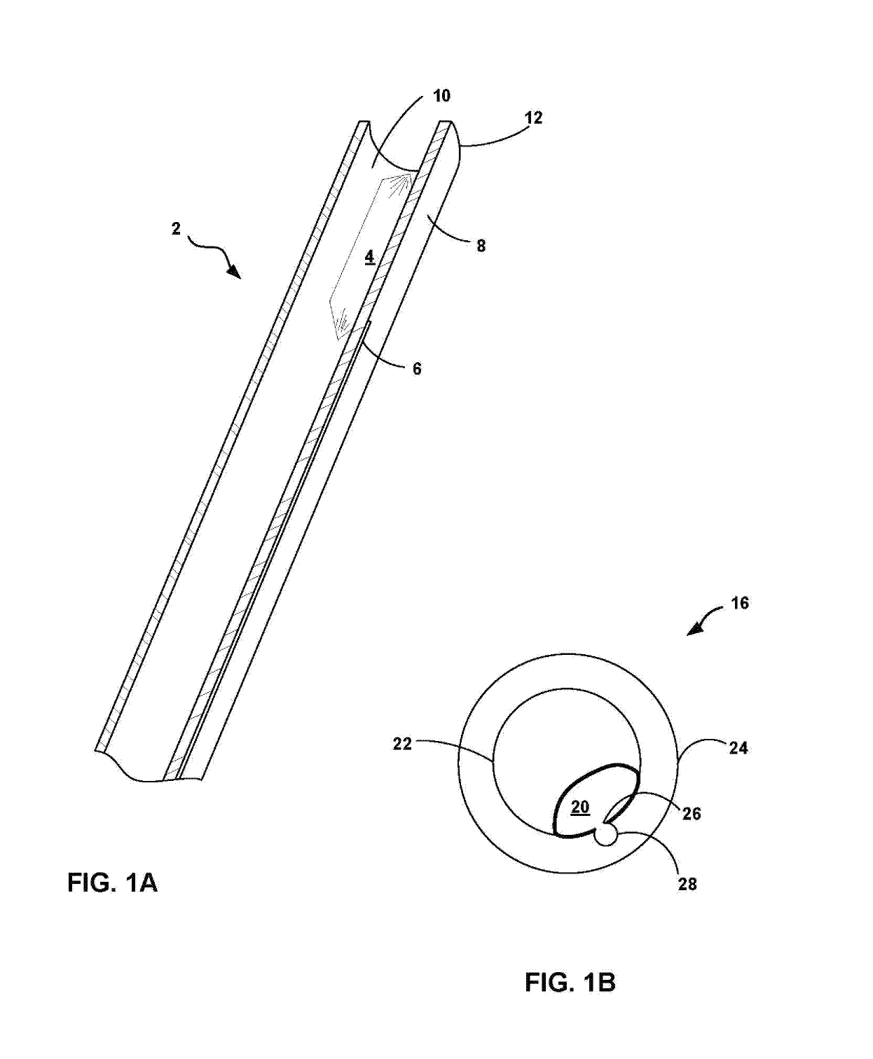

FIG. 1A is a conceptual diagram illustrating an example of a catheter 2 with an expanding member 4 attached to the inner wall of catheter 2, in accordance with one or more techniques of the present disclosure. In some examples, catheter 2 may be a guide catheter suitable for receiving guide wires, microcatheters, and other medical devices. Examples of medical devices may include, but are not limited to, stents, balloons, valves, sensors or other type of devices that a physician may deliver through the vascular system of the patient during the procedure for treating the patient's cardiovascular disease. Expanding member 4 may comprise a balloon and an inflation port. As shown in the example of FIG. 1, the view of catheter 2 is a cross sections view where catheter 2 was cut longitudinally. Catheter 2 may include an inner wall 10, an outer wall 8, and the balloon (i.e., expanding member 4), which may inflate through an inflation lumen 6. Multiple layer configurations, in some examples, may include layers for affecting the rigidity of catheter 2. The layers may be made out of similar or different materials, to increase or decrease the rigidity of catheter 2, increasing or decreasing the catheter flexibility relative to the center longitudinal axis. In other examples, certain layers may be made of materials that affect the torsional rigidity of the catheter, making catheter 2 flexible enough for navigating through blood vessels, but sufficiently rigid preventing catheter 2 from kinking. In addition to the layers between inner wall 10 and outer wall 8 being made of different materials, different portions of the distal portion may be made of different materials. For example, the tip at distal end 12 may comprise a softer material, so that catheter 2 does not injury or damage the blood vessels as the physician pushes catheter through towards the coronary artery. In other examples, the layers of catheter 2 may include chemical compounds or material treatments for treating cardiovascular disease.

Catheter 2 may comprise a proximal portion and a distal portion. The distal portion extends from distal end 12, extending longitudinally to approximately half way down catheter 2. Catheter 2 may also include an expanding member 4 that is located anywhere along the distal portion of catheter 2. Catheter 2 may have a design that includes a generally cylindrical or tubular shape. The inside of catheter 2 may have an inner wall 10 extending circumferentially around the inner portion of catheter 2, so that, for example, inner wall 10 is radially distant from the longitudinal axis of catheter 2, creating a lumen, or a hollow shaft, that is the inner portion of catheter 2. In some examples, the exterior of catheter 2 may include an outer wall 8. In the example of FIG. 1A, an expanding member 4 may be attached to inner wall 10 at the distal portion of catheter 2. The expandable member may have a balloon and an inflation port at an inflation portion of the balloon. The balloon may attach to inner wall 10 at the inflation port. In some examples, the balloon may attach directly to inner wall 10. In other examples, the balloon may not attach to inner wall 10, but may be in fluid communication with inflation lumen 6 through an inflation port, so that inflation fluid may flow from the balloon through the inflation port and into inflation lumen 6. The balloon may attach with adhesives, crimped, formed as one piece, or other known ways in the art of medical device equipment. In other examples, the balloon may remain in place by a hypotube that may connect the balloon to the inflation port through inner wall 10 of catheter 2, and in some examples, also through an outer wall. In FIG. 1A, one example is the position of expandable member 4 near distal end 12 of catheter 2. This may be, for example, near the tip of the catheter. In other examples, expandable member 4 may be inches down the inner wall from distal end 12. In yet other examples, expandable member 4 may be anywhere along the distal portion of catheter 2. The distal portion of catheter 2 may comprise distal end 12, and in some examples the distal portion may also comprise a tip of the catheter, and may extend to the proximal portion of catheter 2.

In some examples, the distance between inner wall 10 and outer wall 8 may vary depending on the different materials used to form catheter 2 and the configuration of catheter 2. Catheter 2 may be made from materials that increase the rigidity of catheter 2. In other examples, the materials forming catheter 2 may increase rigidity so that the diameter of inner wall 10 remains substantially unchanged when expandable member 4 is in an expanded state. In the example of expandable member 4 having a balloon, inflation lumen 6 may provide a pathway for inflation fluid, such as saline or IV contrast medium, to be delivered to the balloon through the inflation port. When the inflation fluid is injected into the inflation lumen 6, expandable member 4 fills with the inflation fluid, and may consequently, expand open.

During expansion, the balloon of expandable member 4 may increase, becoming larger in diameter from the contracted state, and in one example, may expand radially so that expandable member 4 contacts inner wall 10 opposite inflation lumen 6. The balloon of expandable member 4 may expand in different directions. In some examples, the balloon of expandable member 4 may expand radially, while in other examples, the balloon of expandable member 4 may expand longitudinally, or both. The balloon of expandable member 4 may fill with inflation fluid and expand radially, increasing the diameter, and thus, occupying the area within catheter 2 sufficient enough to contact a guide wire or other medical equipment that may be present within the inner shaft portion of catheter 2 when expandable member 4 expands. When expandable member 4 receives inflation fluid to increase the volume of the balloon of expansion member 4 sufficient enough to expand expandable member 4 such that the exterior surface of the balloon may contact inner wall 10.

As expandable member 4 continues to expand, the increase of inflation fluid inside expandable member 4 increases the radial pressure or force outward such that the radial force from inner wall 10 of catheter 2 outward, i.e., towards exterior wall 8. In some examples, when the balloon of expandable member 4 fills with inflation fluid and expands radially outward, filling the inner lumen of catheter 2, catheter 2 does not expand radially. In this example, the diameter of catheter 2 does not increase at least the portion of catheter 2 extending around expandable member 4, even when the balloon of expanding member 4 is in the expanded state. In this example, another medical device may be in the inner shaft of catheter 2, so that at least a portion of the medical device is across from expandable member 4. The medical device may be at least one of a guide wire, microcatheter, or any other medical equipment being inside catheter 2 near expandable member 4 during inflation. In this position, when the balloon of expandable member 4 expands radially, the balloon may push the medical device (e.g., the guide wire) against inner wall 10. The medical equipment may remain held against inner wall 10 when expandable member 4 holds that the same volume of inflation fluid or has an increase in the volume of inflation fluid. In some examples, expandable member 4 holds the guide wire in place against inner wall 10 with sufficient force to limiting the motion of the guide wire. In this example, the diameter of catheter 2 remains substantially unchanged as increasing inflation fluid volume increases the force of the guide wire against inner wall 10. In one example, a physician may reduce the inflation fluid within expandable member 4, causing the pressure of the balloon of expandable member 4 to reduce, and consequently, reduce the force holding the guide wire against inner wall 10. When enough inflation fluid is removed, the guide wire may regain movement.

Prior to injection, expandable member 4 may be in a contracted state, where expandable member 4 does not contain inflation fluid, or returns to the contracted state after expansion. To collapse down to return to the contracted state, a physician or medical assistant may remove the inflation fluid (and any air or substance) in inflation lumen 6 or expandable member 4 using suction device (e.g., syringe) or other removal techniques. Emptying expandable member 4 may allow expandable member 4 to contract, reducing the diameter from the expanded diameter, when expandable member 4 is in the contracted state. The reduced diameter may allow sufficient space in the inner hollow of catheter 2 for guide wires, microcatheters, and other medical devices that the physician may utilize during cardiovascular procedures to fit through catheter 2, advancing past expandable member 4, and possibly rotate as needed during the procedure. In some examples, expandable member 4 may have radial pressure inward from the structure of expandable member 4 (e.g., a stent or spring like structure), contracting expandable member 4 into a contracted state. In other examples, removing the inflation fluid from expandable member 4 and inflation lumen 6, and any air or other substance, reduces radial force outward. In other examples, expandable member 4 contracts sufficiently to the contracted state, allowing medical equipment to fit through catheter 2, for example, over the wire medical devices that are delivered to the coronary artery via a guide wire or microcatheter. In some examples, despite the changing diameter of expandable member 4, the diameter of catheter 2 remains substantially unchanged as expandable member 4 goes from contract state to expanded state and back to the contracted state.

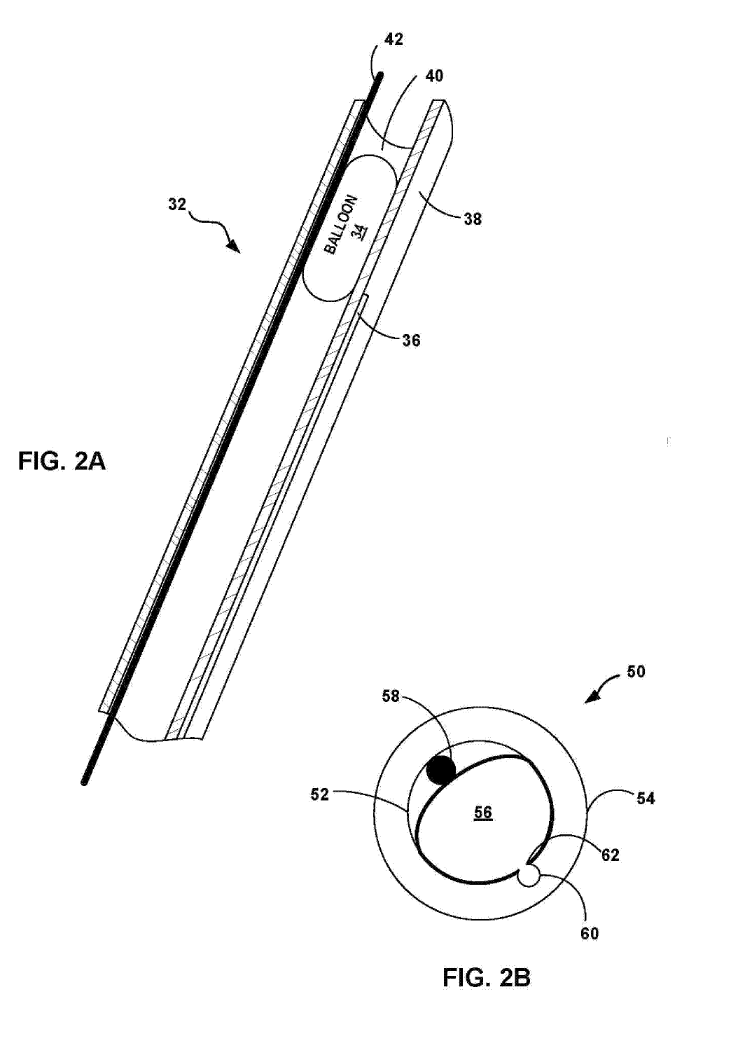

In other examples, expandable member 4 may contract to the contracted state by other means, using means to increase the radial force inward with a structure that contracts the material of expandable member 4 back to the contracted state, or a different flattened shape from the original contracted state, forcing inflation fluid to leave expandable member 4 and sufficient enough to allow the guide wire or other medical equipment to move again within catheter 2. Expansion of expandable member 4 is further with the description of examples of FIGS. 2A-B. FIG. 1B is an example of a cross sectional view of a catheter 16, illustrating the expandable member being a balloon 20 with an inflation port 26. The expandable member may also have an open portion, which is an opening of the balloon and is in fluid communication with an inflation lumen 28. Inflation fluid can flow into balloon 20 from inflation lumen 28. In this example, balloon 20 is in the contracted state and inflation port 26 couples inflation lumen 28 with balloon 20. Inflation port 26 is the portion where fluid flows from inflation lumen 28 into balloon 20. In some examples, inflation port 26 may be an opening in inner wall 22. Inflation port 26 may comprise a valve controlling fluid flow. In other examples, inflation port 26 may be an opening allowing free fluid flow, for example, open without a valve or parts blocking flow. In, yet other examples, inflation port 26 may be a connection, part connecting balloon 20 to inflation lumen 28. The connection part may also, in some examples, also have flow regulation means controlling inflation fluid flowing from inflation lumen 28 to balloon 20.

In another example, FIG. 1B shows a balloon attached to an inner wall 22 of catheter 16, where the attachment limits the movement of balloon 20, particularly at the point of connection between balloon 20 and inner wall 22. Due to the attachment to inner wall 22, balloon 20 remains in position at inflation port 26. In another example, the balloon itself may not be attached to the inner wall of the catheter, but the balloon remains in place, because of coupling to the inflation port. In this example, the inflation port may connect to the balloon at one end, and the inflation port may connect to the inner wall of the catheter at a second end. In some examples, the contact area between balloon 20 and inner wall 22 remains substantially the same even during a procedure when a physician uses guide wires, and other equipment in the distal portion of catheter 16 that may contact balloon 20. During inflation, balloon 20 may expand and fill with inflation fluid, so that expanded balloon 20 fills at least a portion of the hollow inner lumen of catheter 16. However, the opening, or inflation port 26, between balloon 20 and inflation lumen 28 remains substantially the same, and remains at the same location on inner wall 22 of distal portion of catheter 16. In other examples, balloon 20 may be a different type of expanding device that would secure a guide wire or other medical equipment against an inner wall 22 when expanded.

In another example, catheter 16 may have an inflation lumen 28, which is a pathway for inflation fluid to flow from the proximal portion of catheter 16 to the distal portion of catheter 16. Inflation fluid flows from inflation lumen 28 into balloon 20 via inflation port 26, filling and consequently, expanding balloon 20. Inflation lumen 28 may be substantially tubular in shape. In some examples, the tubular shape may be a pre-constructed member located between inner wall 22 and outer wall 24. In other examples, inflation lumen 28 may be a path formed from a gap in the material between inner wall 22 and outer wall 24, forming a hollowing or a lumen. In these examples, inflation lumen 28 allows fluid to flow longitudinally (see e.g., FIG. 1A) through catheter 16 between inner wall 22 and outer wall 28 to balloon 20. In yet another example, inflation lumen 28 may lie within part of outer wall 28 or exterior to outer wall 28 of catheter 16. In this example, a lumen may connect inflation lumen 28 to balloon 20 through an opening in both inner wall 22 and at least a portion of outer wall 24, allowing inflation fluid to flow to balloon 20. Various devices, including but not limited to syringes or inflation tools may be attached the proximal port allowing for controlled insertion and removal of fluid into the inflation lumen to allow to inflation and deflation of the balloon at the distal end of the inflation lumen.

In some examples, inflation fluid may flow from balloon 20 to inflation lumen 28, which may channel the inflation fluid from the distal portion of catheter 16 to the proximal portion of catheter 16. The flow of inflation fluid out of balloon 20 and towards the proximal portion of catheter 16 may result from forces acting on balloon 20, resulting in balloon 20 contracting from an expanded state to, for example, the contracted state. The physical force of balloon 20 contracting may displace the inflation fluid within balloon 20, flowing through the pathway at inflation port 26 and may continue through inflation lumen 28. In another example, the physician performing the procedure may contract the inflated balloon back to the contracted state by removing inflation fluid from the inflation lumen 28 and balloon 20 with a suction device. Techniques for inflation fluid removal may include connecting an empty syringe or suction device to the proximal portion of catheter 16, at an opening opposite the coronary arteries. The syringe creates a vacuum so that pulling or sucking inflation fluid out of the inflation lumen 28 and balloon 20 and any areas there between (e.g., inflation port 26). The force of the suction from removing the inflation fluid may collapse balloon 20. The medical device of claim 2 wherein the balloon being constructed and arranged for expanding from a contracted state to an expanded state, wherein the inner wall of the catheter maintains a diameter when the balloon expands.

In other examples of FIG. 1B and FIG. 1A, inflation lumen 28 extends longitudinally from balloon 20 (balloon 4 of FIG. 1A) on the distal portion of catheter 16 to at least a portion of the proximal portion of catheter 16. Inflation lumen 28 may extend along the inner wall of catheter 16 before connecting with balloon 20. For example, inflation lumen 28 may extend (e.g., 250 cm to 400 cm) along inner wall 22 to a proximal end of inflation lumen 28. The proximal end of inflation lumen 28 may be, for example, at a proximal opening of the distal portion at the proximal end of the catheter. The proximal opening may have a port, seal, or valve to control flow, such as inflation fluid flow, in and out of inflation lumen 28.

A catheter comprising a proximal portion, a distal portion and an expandable member located at the distal portion, said expandable member being constructed and arranged for expanding at least a portion of the expandable member from a contracted state to an expanded state. The expandable member having an inflation port for receiving an inflation fluid therein. The expandable member comprises a balloon and the inflation port. The inflation port is connected to an inflation lumen and communicating with the balloon through said inflation port. The distal portion of the catheter having an inner wall and an outer wall, said expanding member attached to said distal portion at the inner wall. The catheter may be a guide catheter. The expandable member comprises a balloon and the inflation port at an inflation portion of the balloon, wherein the balloon being coupled to the inner wall at the inflation port. The expandable member further comprises an inflation lumen associated with an inflation fluid, said inflation lumen in fluid communication with the balloon through said inflation port. The balloon being constructed and arranged for expanding from a contracted state to an expanded state, wherein the inner wall of the catheter maintains a same (or substantially unchanged) diameter when the balloon expands. In other examples, the inflation lumen is exterior of the balloon. The inflation lumen extends longitudinally from the balloon on the distal portion of the catheter to at least a portion of the proximal portion of the catheter. The balloon being constructed and arranged for contracting from the expanded state to the contracted state. The balloon being constructed of a material which resiliently deforms under radial pressure or removal of the inflation fluid. The inner wall of said catheter has a diameter large enough to receive at least one of a guide wire, a microcatheter, or other medical equipment when the balloon is in the contracted state. The balloon in the expanded state is at least partially inflated creating radial pressure to hold the at least one of the guide wire, the microcatheter, or the other medical equipment between the balloon and the inner wall in a substantially stationary position. The catheter being constructed with multiple layers between the inner and outer walls. In some examples, the inflation lumen being positioned between the inner wall and the outer wall. In other examples, the inflation lumen being positioned exterior to the outer wall.

The balloon is attached at a portion of catheter between a first point at the tip of the catheter on the proximal end and a second point that is located half way between the proximal and distal ends of the catheter. In other examples, a method for using the catheter may comprise the steps of inserting a catheter in an artery of a patient, wherein the catheter has an inner and outer wall, inserting medical equipment in the catheter, and inflating a balloon attached to a first portion of the inner wall of the catheter. The method may further comprise the step of contacting the medical equipment with the inflated balloon, wherein the medical equipment contacts a second portion of the inner wall of the catheter different from the first portion of the inner wall of the catheter where the balloon is coupled to the inner wall. The inflating the balloon may limit the motion of the medical equipment.

In other examples, this disclosure relates to a medical equipment securing system comprising a catheter that has a proximal portion, a distal portion, and an expandable balloon located at the distal portion. The balloon being arranged for expanding from a contracted state to an expanded state and is adapted for expansion from a distal end of the catheter. The balloon having an inflation port at a distal portion thereof. The system may also have an inflation lumen in fluid communication with the inflation port for supplying an inflation fluid to the balloon. The system may further comprising at least one of a guide wire, a microcatheter, and a medical device, being sized to slide through the catheter, wherein the balloon in the expanded state limits the motion of the at least one of said guide wire, microcatheter, and medical device.

The balloon of the expandable member of this disclosure may be constructed of a material which resiliently deforms (e.g., compresses and bulges) under radial pressure. For example, FIG. 2A illustrates balloon 34 and FIG. 2B illustrates balloon 56, which may be made of a polymer (e.g. silicone rubber), polyethylene, polypropylene, polyurethane or nylon. Balloon 34 may be 10-20 mm long for example. These dimensions, however, are merely representative for illustrative purposes only and are not meant to be limiting. Balloon 34 is attached to the inner side of a catheter 32, e.g., a guide catheter, at a portion of balloon 34 near the connection to inflation lumen 36 so that balloon 34 may expand and contract, but remain stationary at the area balloon 34 connects to catheter 32. In accordance with this disclosure, balloon 34 or 56, as illustrated in FIGS. 2A and 2B, is included inside catheter 32 or catheter 50. FIG. 2A is an example of balloon 34 expanding within the inner lumen (formed by inner wall 40) of catheter 32. Balloon 34 may expand due to inflation fluid into balloon 34 that creates radial pressure and causes balloon 34 to expand. Balloon 34 has a diameter that is relative to catheter 32 and wide enough to hold a guide wire 42 or other medical equipment securely against an outer wall 38. In FIG. 2B, balloon 56 is a cushion and/or substrate of enlarged diameter relative to catheter 50 to expand and hold guide wire 58 during the delivery procedure or device exchange/change procedure. When balloon 34 is in a contracted state or partially expanded (inflated) state, guide wire 58 is longitudinally movable (e.g., proximally or distally from the position shown in FIG. 2A) within catheter 32, balloon 34 remains attached to catheter 32. Balloon 34 is fixed to inner wall 40 and has limited motion even when guide wire 42 moves. In the example of FIG. 2A, balloon 34 expands and, once the diameter of balloon 34 is near the diameter of inner wall 40, balloon 34 holds and guide wire 42 against inner wall 40. In the examples of FIGS. 2A and 2B, balloon 34 and 56 may be cylindrical in form relative to the shape of the inner lumen (or inner wall 40) of catheter 32, providing an enlarged area or portion for holding guide wire 34 at inner wall 40 when balloon 34 is in an expanded state. In this example, balloon 34 remains fixed to inner wall 40 during expansion and contraction.

In other examples, a marker band may also be included on balloon 34, allowing better imaging of balloon 34 to determine the diameter during expansion and whether guide wire 42 was secured against inner wall 40. Any radiopaque material such as gold is useful for this purpose. During a cardiovascular procedure, a guide catheter may advance through a patient's vasculature adjacent or near to a targeted treatment area. After guide wire 42 is advanced through catheter 32, other medical devices may advance along guide wire 42. For example, medical devices may be added during a procedure, such as a stent and/or balloon that may advance along guide wire 42 for treating a diseased vascular area, and in other examples, a physician may need to exchange medical devices during a procedure for a different size. To improve safety and ease during the vascular procedure, balloon 34 inflates, expanding to a larger diameter in an expanded state and filling a portion (e.g., radially and longitudinally) of the hollow inner area of catheter 32 with balloon 34, and the inflation limits the movement of guide wire 42 by holding guide wire 42 against inner wall 40. Balloon 34 may hold guide wire 42 in a fixed position while it is sufficiently inflated. When the physician no longer needs guide wire 42 to be held in place, balloon 34 may deflate, collapsing balloon 34 to a reduced size and shape with a smaller diameter, so that guide wire 42 may move or other objects may move through catheter 32.

In the examples of this disclosure, guide wire 42 is only one example and balloon 34 may expand at least partially to hold at least one medical device, such as a guide wire 42, a microcatheter, or another medical device in a fixed position in catheter 32 between balloon 34 and inner wall 40. The radial pressure or force extending outward to inner wall 40 from expanded balloon 34 is sufficient to hold any of these medical devices in a substantially stationary position, as illustrated, for example, in FIG. 2A. In the example of FIG. 2B, guide wire 58 is held against inner wall 52 of catheter 50 by balloon 56. Inner wall 40 may be of a stiffer material, so that it does not deform radially when balloon 56 expands. In some examples, there is at least one layer, and in some instances multiple layers, between inner wall 40 and outer wall 54. The layers may form a stiffer catheter 50 that minimally expands in diameter, but may be flexible for advancing through the vascular system. Balloon 56 may, in some examples, form around at least a part of guide wire 58 as balloon 56 expands due to radial force outward towards inner wall 52. In other examples, balloon 34 may be a different expanding device that is not a balloon. The expanding device may increase in diameter so that the expanding device holds the medical device (e.g., guide wire 42, a microcatheter, etc.) in place, such as against inner wall 40, limiting the motion of the medical device (e.g., guide wire 42).

FIG. 2A illustrates an inflation lumen 36 between inner wall 40 and outer wall 38. FIG. 2B is a cross sectional view of catheter 32 of FIG. 2A. FIG. 2B is a conceptual diagram illustrating an example of another view of the inflated balloon attached to inner wall 52 of the catheter, in accordance with one or more techniques of the present disclosure. Inner wall 52 forms inner side of catheter 50, extending circumferentially such that inner wall 52 forms a hollow inner lumen of catheter 50, where balloon 56 (or inflatable member) may attach to. The section of catheter 50 may, as an example, show the cross sectional view of an inflation lumen 60. In FIG. 2B, inflation port 62 may be an opening between balloon 56 and inflation lumen 60, where a portion of balloon 56 connects to inner wall 52 near inflation lumen 60. In some examples, inflation port 62 may be a valve or other formed connection part connecting to balloon 56 at one portion and inflation lumen 60 at a different portion. In other examples, the connection at inflation port 62 is due to crimping, adhesion, or integral connection. At inflation port 62, the connection of balloon 56 may limit the motion of balloon 56, at least at the connection portion of balloon 56, so that balloon 56 may expand radially while holding position at the connection. In some examples, balloon 56 (or any expandable member represented by balloon 56) may expand in a cylindrical shape, so that the balloon has an inner shaft. The inner shaft allows fluid to flow through the shaft. In one example, blood flowing through the vascular system may also flow through balloon 56, so that flow is not critically reduced during a procedure. In other examples, catheter 32 may have safety features limiting the movement proximally and distally of balloon 34 should the connection between balloon 34 and inner wall 40 cease. For example, the distal end of distal portion of catheter 32 may form a narrowing or have protrusions as a safety mechanism, so that balloon 34 does not exit the balloon is the connection portion of balloon 34 to inner wall 40 ceases and balloon 34 moves longitudinally

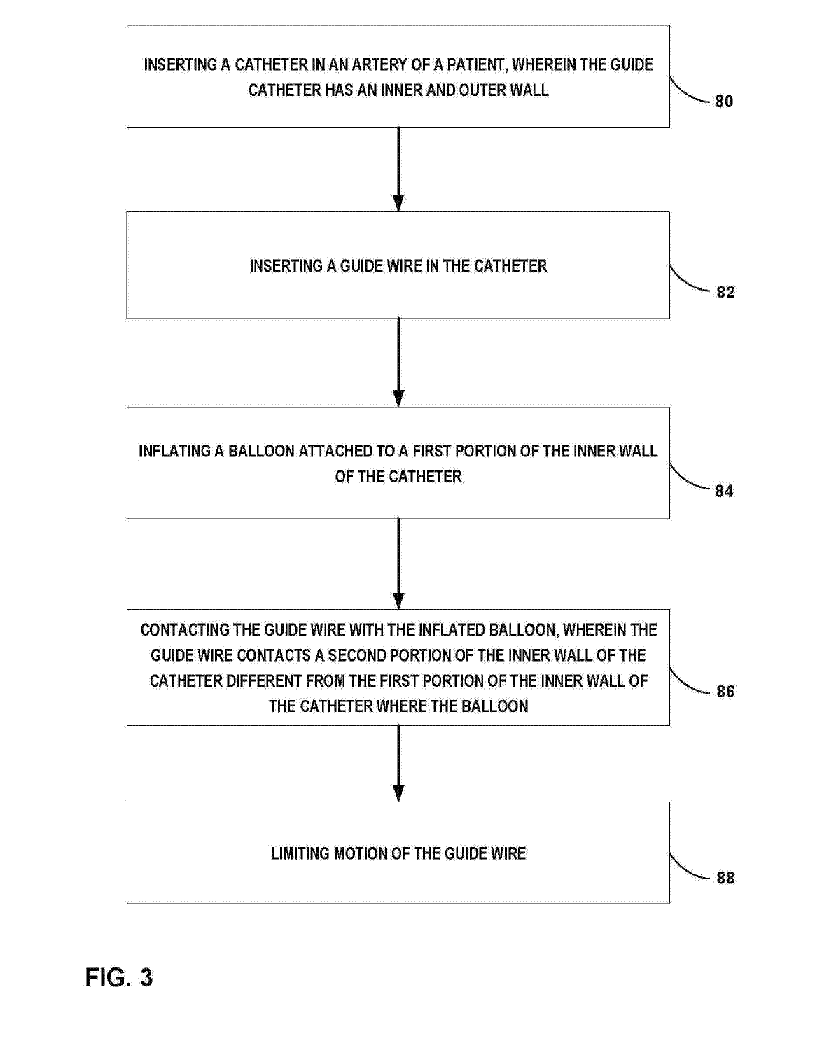

FIG. 3 is a flow diagram illustrating example steps of the method for using a catheter with a balloon attached to the inner wall of the catheter, in accordance with one or more techniques of the present disclosure. For example, the method may include inserting a catheter (e.g., a guide catheter) into an artery of a patient, wherein the catheter has an inner and outer wall (80). The catheter may further have a balloon attached to a distal portion of the inner wall of the catheter. The method may further include inserting a guide wire in the catheter (82), and when a portion of the guide wire is next to the balloon in the catheter, inflating a balloon attached to a first portion of the inner wall of the catheter (84). The balloon may inflate with inflation fluid injected into an inflation lumen, which is in fluid communication with the balloon. The attachment of the balloon to the first portion of the inner wall may limit the motion of the balloon at the area of attachment. After inflation, the steps may include the inflated balloon may contact the guide wire, wherein the guide wire contacts a second portion of the inner wall of the catheter different from the first portion of the inner wall of the catheter where the balloon attaches to the inner wall (86). For example, the second portion of the inner wall may be at any point along that inner wall other than where the balloon attaches to the inner wall of the catheter and the inflation port. In one example, the second portion may be directly across from the inflation port, however, because the guide wire may move until the balloon expands securing the balloon against the inner wall, the guide wire may move to a different portion of the inner wall that is not directly opposite and the balloon secures the guide wire at that location.

In other examples, the balloon expands due to radial pressure as the balloon fills with inflation fluid. When a guide wire is within an inner shaft portion of the guide catheter at the time when the balloon expands, the balloon may expand enough so that the guide wire presses against the inner wall of the guide catheter. When the balloon contains enough inflation fluid, the radial pressure is great enough such that the balloon presses the guide wire against the catheter's inner wall sufficient enough so that the position of the guide wire sandwiched between the balloon and inner wall limits that movement of the guide wire. In this example, the guide wire was included, however, instead of the guide wire, a microcatheter or other medical device may be present and secured against the inner wall of the catheter by the inflated balloon. Additionally, the balloon may secure more than one guide wire, microcatheter, or other medical device against the inner wall of the catheter. These devices may be small enough to fit into the inner shaft of the guide catheter, so that the physician may perform the cardiovascular procedure on the patient.

The balloon inflated due to inflation fluid entering in the balloon through an inflation lumen. The inflation lumen connects to the inflation port, which may further connect to the balloon. The inflation port has an opening so that inflation fluid flows from the inflation lumen through the inflation port and into the balloon. Thus, the inflation lumen is in fluid communication with the inflation port, and in some examples the balloon, for supplying an inflation fluid to the balloon, wherein said inflation lumen is positioned between an inner wall and an outer wall of said catheter. In other examples, the inflation lumen may be located outside of the outer wall of the guide catheter.

The disclosure describes using the guide catheter with a balloon for coronary procedures. However, using a guide catheter as described in this disclosure may also be applicable for other non-coronary medical procedures that use guide catheters and/or guide wires. Various examples have been described. These and other examples are within the scope of the following claims.

What is claimed is:

1. A medical device comprising:
a catheter comprising a proximal portion, a distal portion, and an expanding member located at the distal portion, said distal portion having an inner wall and an outer wall, said expanding member attached to said distal portion at the inner wall.

2. The medical device of claim 1 wherein the expandable member comprises a balloon and an inflation port at an inflation portion of the balloon, wherein the balloon being coupled to the inner wall at the inflation port.

3. The medical device of claim 2 wherein the expandable member further comprises an inflation lumen associated with an inflation fluid, said inflation lumen in fluid communication with the balloon through said inflation port.

4. The medical device of claim 2 wherein the balloon being constructed and arranged for expanding from a contracted state to an expanded state, wherein the inner wall of the catheter maintains a diameter when the balloon expands.

5. The medical device of claim 3 wherein the inflation lumen is exterior of the balloon.

6. The medical device of claim 3 wherein the inflation lumen extends longitudinally from the balloon on the distal portion of the catheter to at least a portion of the proximal portion of the catheter.

7. The medical device of claim 3 wherein the balloon being constructed and arranged for contracting from the expanded state to the contracted state.

8. The medical device of claim 3 wherein the balloon being constructed of a material which resiliently deforms under radial pressure or removal of the inflation fluid.

9. The medical device of claim 3 wherein the catheter is a guide catheter.

10. The medical device of claim 4 wherein the inner wall of said catheter has a diameter large enough to receive at least one of a guide wire, a microcatheter, or other medical equipment when the balloon is in the contracted state.

11. The medical device of claim 10 wherein the balloon in the expanded state is at least partially inflated creating radial pressure to hold the at least one of the guide wire, the microcatheter, or the other medical equipment between the balloon and the inner wall in a substantially stationary position.

12. The medical device of claim 11 wherein the catheter being constructed with multiple layers between the inner and outer walls.

13. The medical device of claim 12 wherein the inflation lumen being positioned between the inner wall and the outer wall.

14. The medical device of claim 12 wherein the inflation lumen being positioned exterior to the outer wall.

15. The medical device of claim 12 wherein the balloon is attached at a portion of catheter between a first point at the tip of the catheter on the proximal end and a second point that is located half way between the proximal and distal ends of the catheter.

16. A method comprising:
inserting a catheter into an artery of a patient, wherein the catheter has an inner and outer wall;
inserting medical equipment in the catheter; and
inflating a balloon attached to a first portion of the inner wall of the catheter.

17. The method of claim 16, further comprising the step of contacting the medical equipment with the inflated balloon, wherein the medical equipment contacts a second portion of the inner wall of the catheter different from the first portion of the inner wall of the catheter where the balloon is coupled to the inner wall.

18. The method of claim 16, wherein inflating the balloon limits the motion of the medical equipment.

19. A medical equipment securing system comprising:
a catheter comprising a proximal portion, a distal portion, and an expandable balloon located at the distal portion, said balloon being arranged for expanding from a contracted state to an expanded state, said balloon being adapted for expansion from a distal end of the catheter, said balloon having an inflation port at a distal portion thereof; and
an inflation lumen in fluid communication with the inflation port for supplying an inflation fluid to the balloon.

20. The medical equipment securing system of claim 18, further comprising at least one of a guide wire, a microcatheter, and a medical device, being sized to slide through the catheter, wherein the balloon in the expanded state limits the motion of the at least one of said guide wire, microcatheter, and medical device.
